(19) United States
(12) Patent Application Publication
(10) Pub. No.: US 2023/0351907 A1
(43) Pub. Date: Nov. 2, 2023

(54) ARTIFICIAL INTELLIGENCE SYSTEM TO PROVIDE SYNERGISTIC EDUCATION LINKED TO DRAWING SKILL EVALUATION AND ART EDUCATION AND WRITING EDUCATION

(71) Applicant: I-SCREAM ARTS CO., LTD., Seoul (KR)

(72) Inventors: Ji-hoon KIM, Seoul (KR); Seung-ah KIM, Sejong (KR); Jong Min KIM, San Jose, CA (US)

(21) Appl. No.: 18/350,993

(22) Filed: Jul. 12, 2023

Related U.S. Application Data
(63) Continuation of application No. PCT/KR2021/016764, filed on Nov. 16, 2021.

(30) Foreign Application Priority Data
Sep. 3, 2021 (KR) 10-2021-0117708
Nov. 11, 2021 (KR) 10-2021-0154401

(51) Int. Cl. G09B 5/02

(52) U.S. Cl. CPC G09B 5/02

(57) ABSTRACT

An artificial intelligence to provide a synergistic education linked to drawing skill evaluation and an art education and a writing education according to an exemplary embodiment of the present disclosure may include a communication unit, a user interface for providing an interactional environment to a user who is a learner, and receiving, through the communication unit, picture data which is created by a second user who is an educator to provide to the user, an art competency evaluation unit which is trained to present a picture subject about the picture data to the user as a question and evaluate an art competency of the user with an art competency score which is calculated by analyzing whether picture data created by the user is picture data in accordance with the picture subject, a picture similarity evaluation unit which is trained to present the picture data of the second user to the user for guiding the user, extract features of the picture data created by the user and picture data of the second user, and evaluate a picture similarity between the picture data created by the user and the picture data of the second user, and a keyword and feature extraction unit which is trained to analyze a document created by the user to include a text and picture data to perform a synergistic education linked to an art education and a writing education, extract a core keyword in the text and an object of picture data corresponding to the core keyword, and exchange the feedback of the core keyword and the object with the user.

### TECHNICAL FIELD

The present disclosure relates to an artificial intelligence system to provide synergistic education linked to drawing skill evaluation, art education and writing education. More particularly, the present disclosure relates to a technique that evaluates a drawing skill of a user to provide a customized art education curriculum according to an expressive ability level of the user to a user. In addition, the present disclosure relates to improve an expressive ability of the user by allowing an artificial intelligence model to make up for a part with an insufficient expression from a document created by a user through feedback with the user, during a synergistic education process linked to an art education and a writing education.

### BACKGROUND ART

Recently, Google has developed a quick draw which is an online game using an artificial intelligence. Quick draw is an online game that challenges a user who is a player to draw a picture or a doodle of an object or a concept. Then, a trained artificial intelligence model recognizes the picture or the doodle created by the user through an image classification technique to guess which object or concept is expressed by the picture or the doodle. In a curriculum of the art education, it is necessary to set different starting points depending on an art competency of a learner. Until now, an expert in the field of art judged an art competency of the learner by evaluating an artwork such as a picture of the leaner with a subjective judgement. However, the subjective judgement is not reliable because an evaluation is not performed based on objective data, and needs to be improved because it is subject to time and place limitations.

In the meantime, a technique has been developed to evaluate (analyze) which part of the similar image is similar to the correct image by analyzing a correlation of the characteristics after extracting each characteristic of a correct answer image which is a criterion of a correct answer and a similar image similar thereto through deep learning of an artificial intelligence model. An expert (a learning tutor) in the field of art analyzes how similarly the learner draws the guiding picture to determine an educational progress level of the learner. In order to determine this, the expert in the field of art needs to analyze each part of the guiding picture and the picture drawn by the learner one by one so that it is difficult for the learner to receive the quick feedback. Therefore, the improvement is necessary.

In the related art, even though the art education and the writing education of the learner were conducted together in a classroom of the school or a lecture room in the academy, the educator did not separately teach the learner about the intersectional part between the art education and the writing education so that the learner was only provided with the learning effect of each education. Recently, a synergistic education is proposed as a new concept of education method which improves an organic thinking ability by not only effectively improving a learning skill in a field desired by the learner, but also acquiring the knowledge of converged subjects at the same time. The synergistic education is an educational method in which two or more subjects are combined so that the learner is provided with a learning effect through a synergy between the educations by feedback of an intersectional element between the educations.

### DISCLOSURE

### Technical Problem

An object of the present disclosure is to provide an artificial intelligence system which evaluates a drawing skill of a user by means of picture data created by the user using an automated method within a short time without having time and place limitation by learning of an artificial intelligence model to provide a customized art education curriculum according to an expressive ability level to a user.

Further, an object of the present disclosure is to provide an artificial intelligence system which analyzes a precise expressive ability of a user based on a picture similarity after evaluating the picture similarity between picture data of a user which is directly drawn by a user after looking at the picture data of the educator, and picture data of the educator through the learning of the artificial intelligence model.

Further, an object of the present disclosure is to provide an artificial intelligence system which allows an artificial intelligence model to make up for a part with insufficient expression from a document created by a user through the feedback with the user, thereby improving the expressive ability of the user with a synergistic education linked to the art education and the writing education.

Technical objects to be achieved in the present disclosure are not limited to the aforementioned technical objects, and another not-mentioned technical object will be clearly understood by those skilled in the art from the description below.

### Technical Solution

In order to achieve the above-described objects, an artificial intelligence system to provide a synergistic education linked to drawing skill evaluation and an art education and a writing education according to an exemplary embodiment of the present disclosure may include a communication unit; a user interface for providing an interactional environment to a user who is a learner, and receiving, through the communication unit, picture data which is created by a second user who is an educator to provide to the user; an art competency evaluation unit which is trained to present a picture subject about the picture data to the user as a question, and evaluate an art competency of the user with an art competency score which is calculated by analyzing whether picture data created by the user is picture data in accordance with the picture subject; a picture similarity evaluation unit which is trained to present the picture data of the second user to the user for guiding the user, extract features of the picture data created by the user and picture data of the second user, and evaluate a picture similarity between the picture data created by the user and the picture data of the second user; and a keyword and feature extraction unit which is trained to analyze a document created by the user to include a text and picture data to perform a synergistic education linked to an art education and a writing education, extract a core keyword in the text and an object of picture data corresponding to the core keyword, and exchange the feedback of the core keyword and the object with the user.

When a predetermined time elapses after presenting a picture subject for picture data to be generated to the user as a question, the art competency evaluation unit may be trained by a first artificial intelligence model to output, on a display of the artificial intelligence system, a window including a drawing area in which the picture data is generated, a tool used for a user to generate the picture data, and information about a time limit to generate the picture data.

Further, the art competency evaluation unit stores picture data generated on the window when the limit time elapses, labels each object from a combination of objects which form the picture data, and analyzes how much the labeled object is correlated with the picture subject to calculate as an art competency score.

Further, after requesting to evaluate the picture similarity by a user whose art competency score is calculated once or more by the art competency evaluation unit, when the second user accesses a terminal of the second user to respond the request of the picture similarity evaluation of the user, the picture similarity evaluation unit may provide the picture similarity evaluation to the user.

Further, when the second user responds the request of the picture similarity evaluation of the user, the picture similarity evaluation unit may be trained by a second artificial intelligence model to display the picture data of the second user input to the terminal of the second user in a first picture area while responding the request by the second user, and output a window in which a tool used for the user to generate the picture data is included in a tool area on a display of the artificial intelligence system.

Further, when the user generates picture data in a second picture area, the picture similarity evaluation unit labels each objects which form picture data created by the user and the objects which form the picture data of the second user, and then may evaluate a picture similarity based on how much the objects which form picture data created by the user and the objects which form the picture data of the second user are similar.

Further, when the user whose art competency score is calculated once or more and picture similarity is evaluated by the art competency evaluation unit and the picture similarity evaluation unit inputs or stores the document through the user interface and then requests the synergistic education, the keyword and feature extraction unit may provide the synergistic education through the document to the user in response to the request of the user.

Further, when the document is analyzed to be determined that the text is not included in the document, the keyword and feature extraction unit may be trained by a third artificial intelligence model to request the user to record a voice to explain picture data in the document, and when the voice of the user is recorded, convert the voice of the user into a text, and determine the text as a text in the document.

Further, the keyword and feature extraction unit may be trained by a fourth artificial intelligence model to recognize a text in a document input or stored through the user interface or a text converted from the voice of the user, and then divide the recognized text into the core keyword and a non-core keyword.

Further, the keyword and feature extraction unit may be trained by a fifth artificial intelligence model to detect a position of the object corresponding to the core keyword from the picture data in the document.

### Advantageous Effects

According to the present disclosure, the drawing skill of the user is evaluated within a short time without having time and place limitations to divide an expressive ability level, and a customized art education curriculum according to a user's expressive ability level may be provided to the user.

Further, according to the present disclosure, it is not evaluated whether the picture data of the user is correct or incorrect, according to a determined classification, but it is evaluated how much the user remembers and expresses the picture data of the educator to analyze the precise expressive ability of the user.

Further, according to the present disclosure, during a synergistic education process linked to the art education and the writing education for improving an expressive ability of the user, the artificial intelligence model makes up for a part with an insufficient expression from a document created by a user through feedback with the user to improve the user's expressive ability.

Further, according to the present disclosure, the synergistic education linked to the art education and the writing education gives fun to the user to consistently conduct each of the art education and the writing education, and the learning effect by the synergistic education may be provided.

A technical object to be achieved in the present disclosure is not limited to the aforementioned effects, and another not-mentioned effects will be obviously understood by those skilled in the art from the description below.

### DESCRIPTION OF DRAWINGS

FIG. 1 is a block diagram schematically illustrating an artificial intelligence system according to an exemplary embodiment of the present disclosure.

FIG. 2 is a view illustrating an example of an art competency evaluating process of an art competency evaluation unit illustrated in FIG. 1.

FIG. 3 is a view illustrating an example of a picture similarity evaluating process of a picture similarity evaluation unit illustrated in FIG. 1.

FIG. 4 is a view illustrating an example of a synergistic education process linked to an art education and a writing education of a keyword and feature extraction unit illustrated in FIG. 1.

### BEST MODE

Hereinafter, exemplary embodiments of the present disclosure will be described more fully with reference to the accompanying drawings for those skilled in the art to easily implement the present disclosure. Description of the present disclosure is just an embodiment for structural and functional description so that the scope of the present disclosure is not interpreted to be limited by the embodiment described in the specification. That is, the embodiment may be modified in various forms so that it is understood that the scope of the present disclosure has equivalents which are capable of implementing the technical spirit. Further, it does not mean that the specific embodiment includes the object or effect proposed in the present disclosure or includes only the effect so that it is not understood that the scope of the present disclosure is limited thereby.

In the meantime, meanings of terms described in the present disclosure can be understood as follows.

The terms “first” or “second” are used to distinguish one component from the other component so that the scope should not be limited by these terms. For example, a first component may be referred to as a second component, and similarly, a second component may be referred to as a first component. It should be understood that, when it is described that an element is “connected” to another element, the element may be directly connected to the other element or connected to the other element through a third element. In contrast, it should be understood that, when it is described that an element is directly connected to another element, no element is present between the element and the other element. Other expressions which describe the relationship between components, that is, “between” and “directly between”, or “adjacent to” and “directly adjacent to” need to be interpreted by the same manner.

Unless the context apparently indicates otherwise, it should be understood that terms “include” or “have” indicate that a feature, a number, a step, an operation, a component, a part or the combination thereof described in the specification is present, but do not exclude a possibility of presence or addition of one or more other features, numbers, steps, operations, components, parts or combinations thereof, in advance.

Unless they are contrarily defined, all terms used herein including technological or scientific terms have the same meaning as those generally understood by a person with ordinary skill in the art. Terms which are defined in a generally used dictionary should be interpreted to have the same meaning as the meaning in the context of the related art but are not interpreted as an ideally or excessively formal meaning if it is not clearly defined in the present disclosure.

An artificial intelligence system to provide a synergistic education linked to drawing skill evaluation and an art education and a writing education according to an exemplary embodiment of the present disclosure (hereinafter, referred to as an “artificial intelligence system 100”) is a system which evaluates a drawing skill of the user 1 to provide a customized art education curriculum according to an expressive ability level to the user 1, and allows the artificial intelligence model to make up for a part with an insufficient expression from a document created by a user through a feedback with the user during the synergistic education process linked to the art education and the writing education to improve the user's expressive ability and components therefor are as follows.

The user 1 is not limited to a gender, education, and age, but in the present disclosure, the user may be a kindergartner or an elementary school student who is a child of low education who needs to be provided with the synergistic education linked to the art education and the writing education.

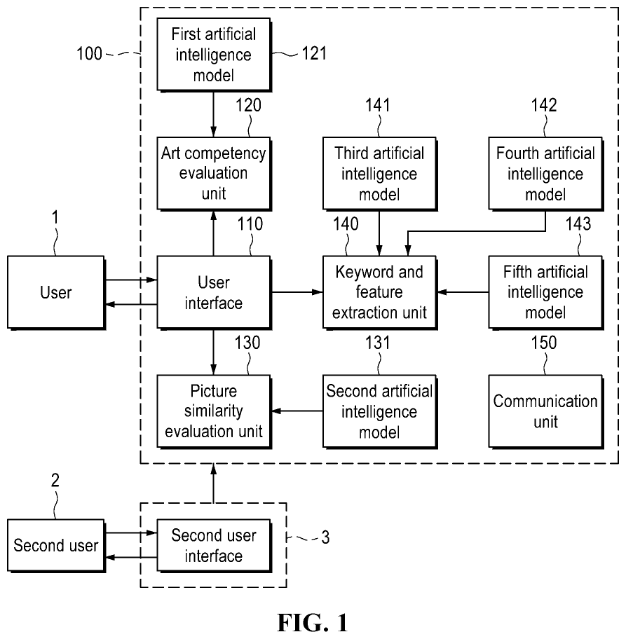

FIG. 1 is a block diagram schematically illustrating an artificial intelligence system according to an exemplary embodiment of the present disclosure.

Referring to FIG. 1, the artificial intelligence system 100 according to the exemplary embodiment of the present disclosure includes a user interface 110, an art competency evaluation unit 120, a picture similarity evaluation unit 130, a keyword and feature extraction unit 140, and a communication unit 150.

The user interface 110 is provided in the artificial intelligence system 100 to provide an interactive environment to the user 1 who is a learner, and provide picture data which is received through the communication unit 150 and is created by a second user 2 who is an educator to the user 1.

The user interface 110 is a concept including a hardware device and a software program to provide an environment in which a user 1 interacts with the artificial intelligence system 100, and to receive an instruction from the user 1 to convert the instruction into electronic data. For example, an input device such as a keyboard, a mouse, and a touch pen, an output device such as a display, and a drawing application which processes data, such as contours and colors, input through the input device to display the data on the output device in real time may be included.

That is, the artificial intelligence system 100 interacts with the user 1 through the user interface 110 so that the picture data 100 may be input from the user 1.

At this time, the picture data is data generated in a picture area of a window output on a display provided in the artificial intelligence system 100 by the user 1, and is formed of a combination of objects generated in a position designated by the user 1. The objects are not limited, but may be information including an item to be evaluated by the art psychological examination.

Here, the art psychological examination may be an HTP test which asks to draw at least one of a house, a tree, and a people which is an object and then analyzes a psychological state of the user from information such as a shape, a color, and a position of the picture. However, the method is not limited so as to evaluate the drawing skill and predict the academic achievement of the user 1 by means of picture data which is formed of a combination of various objects. If the object is a house, as an item constituting the object, house frames (a roof, a wall, and a chimney), windows, and doors may be included, and if the object is a tree, stems, roots, leaves, and fruits may be included.

The art competency evaluation unit 120 is provided in the artificial intelligence system 100 to present a picture subject about the picture data to the user 1 as a question, and analyze whether the picture data created by the user 1 is picture data according to the picture subject, and then evaluate the art competency of the user 1 with a calculated art competency score.

Here, the art competency may include aesthetic sensibility that understands and expresses one's emotion through perception of various objects and phenomena and internalizes aesthetic values while responding to aesthetic experiences. Further, the art competency may include a visual communication ability that understands and interprets image information and visual media from a changing visual culture and visually communicates through creation and criticism using them. Further, the art competency may include a creativity and convergence ability that creatively expresses one's feelings and thoughts and rationally solves various problems confronted during the art activities by linking and fusing art with knowledges and experiences in various fields. Further, the art competency may include an ability to understand an art culture that understands and respects pluralistic values of the art culture with a flexible and open attitude, and a self-initiated art learning ability that develops and reflects on oneself while voluntarily and proactively participating in art activities, and understands, respects, considers, and cooperates the thoughts and feelings of others, during the process.

Further, the art competency evaluation unit 120 is trained by the first artificial intelligence model 121 to evaluate the art competency of the user 1, and the first artificial intelligence model 121 may be trained based on an image classification technique to determine whether the picture data of the user is a picture in accordance with the picture subject.

An example of an art competency evaluating process of a user 1 by the art competency evaluation unit 121 is as follows.

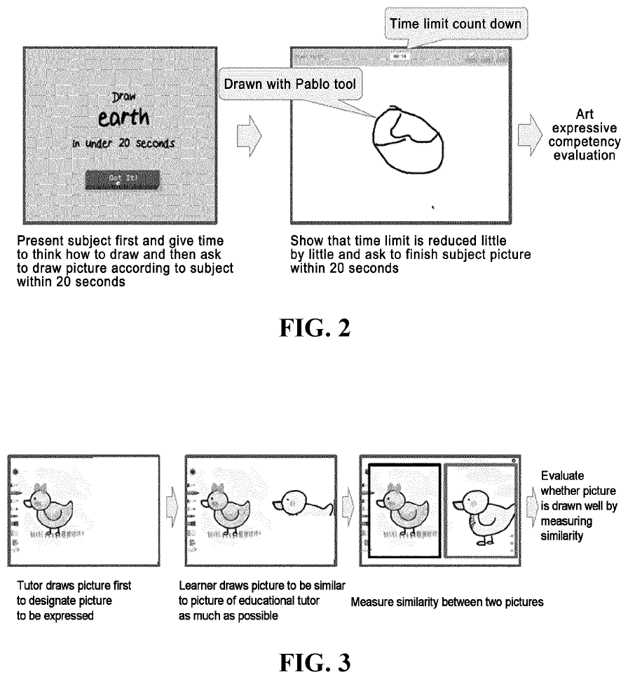

FIG. 2 is a view illustrating an example of an art competency evaluating process of an art competency evaluation unit illustrated in FIG. 1.

Referring to FIG. 2, the art competency evaluation unit 120 is trained by the first artificial intelligence model 121 to present a picture subject about picture data to be created to the user 1 through the display of the artificial intelligence system 100 as a question, and as a predetermined time elapses, output a window including a picture area in which picture data is to be created by the user 1, a tool used by the user 1 to create the picture data, and information about a time limit to create the picture data on the display of the artificial intelligence system 100.

When the time limit elapses, the art competency evaluation unit 120 stores the picture data generated on the window in the database, labels each object from the combination of objects which form the picture data, and calculates an art competency score by analyzing information of each labeled object correlated with the picture subject. At this time, the method of the art competency score is not limited, but may be calculated based on a perfect score of 100 points so that the user 1 who is the child with low education may easily determine the score level.

As described above, the art competency evaluation unit 120 has an advantage in that the art competency of the user 1 may be evaluated using picture data created by the user 1 by an automated method within a short time without having limitations of the time and the place, by training the artificial intelligence model, and the learning level of the user and the effect of the art education may be determined based on the evaluation of the art competency of the user.

In the meantime, the artificial intelligence system 100 includes the art competency evaluation unit 120 to provide an art competency evaluation service to the user 1, and the user 1 may register as a member by generating an ID in various methods to be provided with the art competency evaluation service.

Moreover, when the user 1 logs in with the ID for the first time after registering as a member to the art competency evaluation service, the artificial intelligence system 100 allows the user 1 to take an admission test to evaluate the art competency and periodically take the test for the art competency to determine whether the art competency of the user is improved by the art competency score according to the periodic test.

Referring to FIG. 1 again, the picture similarity evaluation unit 130 is provided in the artificial intelligence system 100 to present the picture data of a second user 2 for guiding the user 1 to the user 1, and then extract features of the picture data created by the user 1 and the picture data of the second user 2 to evaluate the picture similarity between the picture data created by the user 1 and the picture data of the second user 2.

At this time, the user 1 who uses the picture similarity evaluation unit 130 is not limited, but may be a user 1 whose art competency score is calculated once or more by the art competency evaluation unit 120.

Further, the picture similarity evaluation unit 130 is trained by a second artificial intelligence model 131 to evaluate a picture similarity between the picture data created by the user 1 and the picture data of the second user 2, and the second artificial intelligence model 131 may be trained to evaluate the picture similarity by analyzing a correlation of features of the object in every position from a combination of objects which form the picture data created by the user 1 and a combination of objects which form the picture data of the second user 2.

An example of an art similarity evaluating process of a user 1 by the art similarity evaluation unit 130 is as follows.

FIG. 3 is a view illustrating an example of a picture similarity evaluating process of a picture similarity evaluation unit illustrated in FIG. 1.

Referring to FIG. 3, after requesting the evaluation of the picture similarity by the user 1 whose art competency score is calculated once or more through the art competency evaluation unit 120, when the second user 2 accesses the terminal 3 of the second user 2 to respond the request of the evaluation of the picture similarity of the first user 1, the picture similarity evaluation unit 130 responds the request of the second user 2, displays the picture data of the second user 2 which is input to the terminal 3 of the second user in a first picture area by the second user 1, and outputs a window in which a tool used by the user 1 to create the picture data is included in a tool area on the display of the artificial intelligence system.

Thereafter, when the user 1 creates the picture data in a second picture area, after labeling each of the objects which form the picture data created by the user 1 and the objects which form the picture data of the user 2, the picture similarity evaluation unit 130 may evaluate the picture similarity based on how much the objects which form the picture data created by the user 1 and the objects which form the picture data of the second user 2 are similar.

As described above, the picture similarity evaluation unit 130 is trained by the artificial intelligence model to evaluate a picture similarity between picture data of the user 1 which is drawn by the user 1 after looking at the picture data of the second user 2 and the picture data of the educator 2. At this time, the picture similarity evaluation unit may evaluate how much the user 1 remembers and expresses the picture data of the second user, rather than evaluating whether the picture data of the user 1 is correct or incorrect according to a predetermined classification to analyze a precise expressive ability of the user 1.

In the meantime, the artificial intelligence system 100 evaluates the picture expressive ability of the user 1 based on the art competency score calculated by the art competency evaluation unit 120 and the picture similarity evaluated by the picture similarity evaluation unit 130 to identify a picture expressive ability level, and may provide the customized art education curriculum according to a picture expressive ability level to the user 1.

Further, the artificial intelligence system 100 includes the art similarity evaluation unit 130 to provide a memory education service which determines a momentary memory of the user 1 through the degree of the picture similarity to the user 1, and the user 1 may generate the ID in various methods to register as a member to be provided with the memory education service.

At this time, the ID generated by the user 1 may be the ID generated to be provided with the art competency evaluation service.

Moreover, the artificial intelligence system 100 increases the memory education difficulty by reducing a time when the picture data of the second user 2 is displayed in the first picture area, or forming the picture data of the second user 2 with a complex combination of objects, to provide the memory education service according to the levels to the user 1.

Referring to FIG. 1 again, the keyword and feature extraction unit 140 is provided in the artificial intelligence system 100 to analyze a document created by the user 1 to include a text and picture data to extract a core keyword in the text of the document and an object of picture data corresponding to the core keyword, and exchange feedbacks about the core keyword and the object with the user 1.

At this time, the user 1 who uses the keyword and the feature extraction unit 140 is not limited, but may be a user whose picture expressive ability is evaluated by the art competency evaluation unit 120 and the picture similarity evaluation unit 130, and the text in the document and the picture data may have a connection relationship.

Further, when the user 1 requests the synergistic education linked to the art education and the writing education after inputting or storing the document through the user interface 110, the keyword and feature extraction unit 140 provides the synergistic education through the document to the user 1 in response to the request of the user 1.

That is, the keyword and feature extraction 140 may include the text and the picture in the document to provide the synergistic education linked to the art education and the writing education, however, the user 1 may not write the text depending on the education level.

Therefore, the keyword and feature extraction unit 140 is trained by the third artificial intelligence model 141 to record a voice of the user 1 for explaining the picture data in the document to extract a text from a document in which the text is not included, and extract the text from the voice of the user 1.

When the voice of the user 1 is recorded in the keyword and feature extraction unit 140, the third artificial intelligence model 141 may be trained by a speech to text (STT) technique which extracts a text by preprocessing a recording file by removing a noise which is recorded in the recording file together during the process of recording the voice of the user 1, and extracting the voice of the recording file as a text, and then modifying words of the text in accordance with the standard language.

The keyword and feature extraction unit 140 is trained by the third artificial intelligence model 141 to determine a text extracted from the recording file in which the voice of the user 1 is recorded, as a text in the document and recognize the text to extract a core keyword.

That is, the keyword and feature extraction unit 140 recognizes a text in a document input or stored by the user interface 110 or the text extracted from the recording file in which the voice of the user is recorded, and then divides the recognized text into a core keyword and non-core keyword, and is trained by the fourth artificial intelligence model 142 therefor.

The fourth artificial intelligence model 142 may be trained by a technique in which optical character recognition (OCR) and text summarization are merged, to recognize the text and extract the core keyword from the text.

Here, the text summarization may be extractive summarization by which sentences or phrases are extracted from the text to summarize the text, or abstractive summarization by which a new sentence which is not in the text based on the core context of the text to obstructively summarize the text. However, the keyword and feature extraction unit 140 extracts the core keyword from the recognized text so that the extractive summarization may be preferably applied.

In the meantime, the keyword and feature extraction unit 140 may detect a position of the object corresponding to the core keyword extracted by being trained by the fourth artificial intelligence model 142 from the picture data in the document, and may be trained by a fifth artificial intelligence model 143 therefor.

The fifth artificial intelligence model 143 may be trained by object detection, image classification, and image captioning techniques to detect the position of the object (image) corresponding to the core keyword.

Here, the image captioning is a task that looks at the image and explains the image with languages and in the present disclosure, therefore, the image captioning refers to a task that explains objects from the combination of the objects which form the picture data in the document with languages.

Even though it has been described that the keyword and feature extraction unit 140 is trained by the third, fourth, and fifth artificial intelligence models 141, 142, and 143, the third, fourth, and fifth artificial intelligence models 141, 142, and 143 may be implemented by one artificial intelligence model.

An example of a process of providing a synergistic education linked to the art education and the writing education to the user 1 by the keyword and feature extraction unit 140 is as follows.

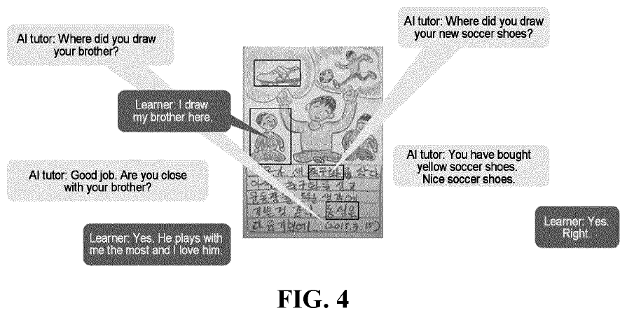

FIG. 4 is a view illustrating an example of a synergistic education process linked to an art education and a writing education of a keyword and feature extraction unit illustrated in FIG. 1. Referring to FIG. 4, the user 1 may input or store, through the user interface 110, a drawing diary which is a document including a text and picture data linked to the text.

Next, the keyword and feature extraction unit 140 extracts a core keyword from the text, and then extracts an object corresponding to the core keyword, and repeats the feedback about the core keyword and/or the object with the user 1 until there is no more core keyword to be extracted to provide the learning effect by the art education and the writing education to the user 1.

As described above, the keyword and feature extraction unit 140 analyzes an intersectional element between the art education and the writing education with a document created by the user 1 through the learning of the artificial intelligence model, and then provides the learning effect to the user 1 based on the synergy between two educations through the feedback with the user 1 about the intersectional element between two educations. In addition, the keyword and feature extraction unit 140 gives fun to the user 1 to consistently conduct the art education and the writing education and may provide the learning effect of the synergistic education linked to the art education and the writing education at the same time.

In the meantime, the artificial intelligence system 100 includes the keyword and feature extraction unit 140 to provide the synergistic education service to the user 1, and the user 1 may register as a member by generating the ID in various methods to be provided with the synergistic evaluation service.

At this time, the ID generated by the user 1 refers to the ID to receive the art competency evaluation service, a memory education service, and a synergistic education service.

The artificial intelligence system 100 includes the keyword and feature extraction unit 140 to make up for a part of an insufficient expression from a document created by a user through feedback with the user 1 during a synergistic education process linked to an art education and a writing education to improve an expressive ability of the user 1.

Moreover, the artificial intelligence system 100 according to the exemplary embodiments of the present disclosure may be implemented by an application or implemented in the form of a program command which may be executed through various computer components to be recorded in a computer readable recording medium. The computer readable recording medium may include solely a program command, a data file, and a data structure or a combination thereof.

Examples of the computer readable recording medium include magnetic media such as a hard disk, a floppy disk, or a magnetic tape, optical recording media such as a CD-ROM or a DVD, magneto-optical media such as a floptical disk, and a hardware device which is specifically configured to store and execute the program command such as a ROM, a RAM, and a flash memory.

As described above, the detailed description of the exemplary embodiments of the disclosed present disclosure is provided such that those skilled in the art implement and carry out the present disclosure. While the disclosure has been described with reference to the preferred embodiments, it will be understood by those skilled in the art that various changes and modifications of the present disclosure may be made without departing from the spirit and scope of the disclosure. For example, those skilled in the art may use configurations disclosed in the above-described exemplary embodiments by combining them with each other. Therefore, the present disclosure is not intended to be limited to the above-described exemplary embodiments but to assign the widest scope consistent with disclosed principles and novel features.

The present disclosure may be implemented in another specific form within the scope without departing from the technical spirit and essential feature of the present disclosure. Therefore, the detailed description should not restrictively be analyzed in all aspects and should be exemplarily considered. The scope of the present disclosure should be determined by rational interpretation of the appended claims and all changes are included in the scope of the present disclosure within the equivalent scope of the present disclosure. The present disclosure is not intended to be limited to the above-described exemplary embodiments but to assign the widest scope consistent with disclosed principles and novel features. Further, claims having no clear quoting relation in the claims are combined to configure the embodiment or may be included as new claims by correction after application.

1. An artificial intelligence system to provide a synergistic education linked to drawing skill evaluation and an art education and a writing education, comprising:
a communication unit;
a user interface for providing an interactional environment to a user who is a learner, and receiving, through the communication unit, picture data which is created by a second user who is an educator to provide to the user;
an art competency evaluation unit which is trained to present a picture subject about the picture data to the user as a question, and evaluate an art competency of the user with an art competency score which is calculated by analyzing whether picture data created by the user is picture data in accordance with the picture subject;
a picture similarity evaluation unit which is trained to present the picture data of the second user to the user for guiding the user, extract features of the picture data created by the user and picture data of the second user, and evaluate a picture similarity between the picture data created by the user and the picture data of the second user; and
a keyword and feature extraction unit which is trained to analyze a document created by the user to include a text and picture data to perform a synergistic education linked to an art education and a writing education, extract a core keyword in the text and an object of picture data corresponding to the core keyword, and exchange the feedback of the core keyword and the object with the user.

2. The artificial intelligence system to provide a synergistic education linked to drawing skill evaluation and an art education and a writing education of claim 1, wherein when a predetermined time elapses after presenting a picture subject for picture data to be generated to the user as a question, the art competency evaluation unit is trained by a first artificial intelligence model to output, on a display of the artificial intelligence system, a window including a drawing area in which the picture data is generated, a tool used for a user to generate the picture data, and information about a time limit to generate the picture data.

3. The artificial intelligence system to provide a synergistic education linked to drawing skill evaluation and an art education and a writing education of claim 2, wherein the art competency evaluation unit stores picture data generated on the window when the time limit elapses, labels each object from a combination of objects which form the picture data, and analyzes how much the labeled object is correlated with the picture subject to calculate as an art competency score.

4. The artificial intelligence system to provide a synergistic education linked to drawing skill evaluation and an art education and a writing education of claim 1, wherein after requesting to evaluate the picture similarity by a user whose art competency score is calculated once or more by the art competency evaluation unit, when the second user accesses a terminal of the second user to respond the request of the picture similarity evaluation of the user, the picture similarity evaluation unit provides the picture similarity evaluation to the user.

5. The artificial intelligence system to provide a synergistic education linked to drawing skill evaluation and an art education and a writing education of claim 4, wherein when the second user responds the request of the picture similarity evaluation of the user, the picture similarity evaluation unit is trained by a second artificial intelligence model to display the picture data of the second user input to the terminal of the second user in a first picture area while responding the request by the second user, and output a window in which a tool used for the user to generate the picture data is included in a tool area on a display of the artificial intelligence system.

6. The artificial intelligence system to provide a synergistic education linked to drawing skill evaluation and an art education and a writing education of claim 5, wherein when the user generates picture data in a second picture area, the picture similarity evaluation unit labels each objects which form picture data created by the user and the objects which form the picture data of the second user, and then evaluates a picture similarity based on how much the objects which form picture data created by the user and the objects which form the picture data of the second user are similar.

7. The artificial intelligence system to provide a synergistic education linked to drawing skill evaluation and an art education and a writing education of claim 1, wherein when the user whose art competency score is calculated once or more and picture similarity is evaluated by the art competency evaluation unit and the picture similarity evaluation unit inputs or stores the document through the user interface and then requests the synergistic education, the keyword and feature extraction unit provides the synergistic education through the document to the user in response to the request of the user.

8. The artificial intelligence system to provide a synergistic education linked to drawing skill evaluation and an art education and a writing education of claim 7, wherein when the document is analyzed to determine that the text is not included in the document, the keyword and feature extraction unit is trained by a third artificial intelligence model to request the user to record a voice to explain picture data in the document, and when the voice of the user is recorded, convert the voice of the user into a text, and determine the text as a text in the document.

9. The artificial intelligence system to provide a synergistic education linked to drawing skill evaluation and an art education and a writing education of claim 8, wherein the keyword and feature extraction unit is trained by a fourth artificial intelligence model to recognize a text in a document input or stored through the user interface or a text converted from the voice of the user, and then divide the recognized text into the core keyword and a non-core keyword.

10. The artificial intelligence system to provide a synergistic education linked to drawing skill evaluation and an art education and a writing education of claim 9, wherein the keyword and feature extraction unit is trained by a fifth artificial intelligence model to detect a position of the object corresponding to the core keyword from the picture data in the document.
